(19) United States
(12) Patent Application Publication
(10) Pub. No.: US 2023/0215518 A1
(43) Pub. Date: Jul. 6, 2023

(54) PER-AND POLYFLUOROALKYL SUBSTANCES (PFAS) MODELING

(71) Applicant: JACOBS ENGINEERING GROUP, INC., Dallas, TX (US)

(72) Inventor: Jeffrey D. Gamlin, Parker, CO (US)

(21) Appl. No.: 18/146,184

(22) Filed: Dec. 23, 2022

Related U.S. Application Data
(60) Provisional application No. 63/295,607, filed on Dec. 31, 2021.

(51) Int. Cl. G16C 20/30; G16C 20/70

(52) U.S. Cl. CPC G16C 20/30; G16C 20/70

(57) ABSTRACT

An exemplary method includes creating a training dataset of per and polyfluoroalkyl substances (PFAS) compound environmental release over time using a computer aided simulation of environmental release of a set of PFAS containing products and training a machine learning model using the training dataset. The method further includes receiving, at the trained machine learning model, environmental data relating to an environment and PFAS concentration data representing PFAS compound concentrations within the environment and generating, using the trained machine learning model, identification of one or more PFAS containing products likely to have caused the PFAS compound concentrations within the environment.

### CROSS REFERENCE TO RELATED APPLICATION

This application claims the benefit of priority to U.S. Application No. 63/295,607, filed Dec. 31, 2021 entitled PER-AND POLYFLUOROALKYL SUBSTANCES (PFAS) MODELING, which is hereby incorporated by reference.

### TECHNICAL FIELD

This application relates to methods and systems for modeling certain man-made compounds, particularly per and polyfluoroalkyl substances (PFAS) compounds.

### BACKGROUND

Per and polyfluoroalkyl substances (PFAS) are a class of thousands of man-made fluorinated compounds, many of which have been identified as toxic and are regulated or soon to be regulated by environmental regulatory agencies. Current talking points in the environmental investigation and remediation industry, where this invention will be used, focus on a narrative that understanding the thousands of PFAS compounds once they are released to the environment is “too complicated” and that we will never figure it all out. Generic methods for understanding the chemical patterns of different PFAS-containing products are in their infancy and might rely on approximately 10 to 20, out of the thousands of PFAS compounds. Predictive modeling of how PFAS are transported, transformed, and attenuated in the environment is almost non-existent at this time, or perhaps rely on only a basic understanding of how approximately 8 to 12 PFAS compounds are transported in the environment, but ignore how they transform along a flow path.

### SUMMARY

An exemplary method includes creating a training dataset of per and polyfluoroalkyl substances (PFAS) compound environmental release over time using a computer aided simulation of environmental release of a set of PFAS containing products and training a machine learning model using the training dataset. The method further includes receiving, at the trained machine learning model, environmental data relating to an environment and PFAS concentration data representing PFAS compound concentrations within the environment and generating, using the trained machine learning model, identification of one or more PFAS containing products likely to have caused the PFAS compound concentrations within the environment.

Additional embodiments and features are set forth in part in the description that follows, and will become apparent to those skilled in the art upon examination of the specification and may be learned by the practice of the disclosed subject matter. A further understanding of the nature and advantages of the present disclosure may be realized by reference to the remaining portions of the specification and the drawings, which form a part of this disclosure. One of skill in the art will understand that each of the various aspects and features of the disclosure may advantageously be used separately in some instances, or in combination with other aspects and features of the disclosure in other instances.

### BRIEF DESCRIPTION OF THE DRAWINGS

The description will be more fully understood with reference to the following figures in which components are not drawn to scale, which are presented as various examples of the present disclosure and should not be construed as a complete recitation of the scope of the disclosure, characterized in that:

FIG. 1 illustrates an example system providing PFAS pattern generation, a PFAS concentration prediction model, and a PFAS product model in accordance with some embodiments of the disclosure.

FIG. 2 is a schematic diagram of an computer system which may be used to implement various embodiments in the examples described herein.

FIG. 3 shows an example framework for representing PFAS containing compounds used in accordance with various embodiments of the disclosure.

FIG. 4 shows an additional example framework for representing PFAS containing compounds and their transformation pathways used in accordance with various embodiments of the disclosure.

FIG. 5 shows example PFAS patterns generated in accordance with various embodiments of the disclosure.

FIG. 6 is a flow diagram of an example process for generating a PFAS pattern in accordance with various embodiments of the disclosure.

FIG. 7 is a flow diagram of an example process for generating predictions of future PFAS concentrations in accordance with various embodiments of the disclosure.

FIG. 8 is a flow diagram of an example process for generating and utilizing a PFAS product model in accordance with various embodiments of the disclosure.

### DETAILED DESCRIPTION

Methods and systems described herein may model PFAS compounds. An understanding of the chemical patterns of different PFAS compounds or PFAS containing products and predictive modeling of how PFAS compounds or PFAS containing products are transported, transformed, and attenuated in the environment may improve investigation and remediation of PFAS compounds by providing an understanding of how the compounds behave once released into the environment. PFAS containing products are common and arise from use by many different industries. For example, carpet coating, cookware coating, textile waterproofing, and aqueous film-forming foam (AFFF) used for firefighting during petroleum or other chemical fires, may all be PFAS containing products. Understanding which PFAS containing products may result in observed PFAS concentrations in an environment may help with investigation and remediation, and provide identification of entities responsible for the observed PFAS concentrations in the environment. Further, knowing which PFAS compounds cause observed environmental conditions may help determine whether remediation is necessary to safely discharge the compounds and prevent contamination of the environment.

Understanding and modeling how PFAS compounds or PFAS containing products behave in the environment is often difficult due to the number of PFAS compounds in existence. For example, it is difficult to categorize the thousands of known PFAS compounds according to how they transform and attenuate once released to the environment. Current understanding of how PFAS compounds behave and transform into one another is uncoordinated and relies on approximately 60 different literature sources. The uncoordinated nature of currently available data makes it difficult to model transformation interactions of PFAS compounds. The PFAS model disclosed herein may use a framework for representing and organizing PFAS compounds to model the compounds, utilizing the available datasets to create a comprehensive framework. The framework may be thought of as a “periodic table of PFAS transformation” and may organize PFAS compounds according to a chemical order.

Interpretation of PFAS analytical data is also difficult, as such analysis is complicated by the chemical composition of PFAS compounds. PFAS compounds include complicated chemical mixtures of hundreds to thousands of individual compounds which change and transform once released into the environment. Further, no consistent data visualization method currently exists. PFAS compounds are often visualized graphically using two-dimensional bar or pie charts without a consistent reference pattern. Further, available models generally include approximately ten to twelve PFAS precursor compounds, where hundreds of PFAS precursors are utilized to understand PFAS transformation and chemical fingerprinting of PFAS compounds. The PFAS model disclosed herein may generate PFAS patterns showing chemical concentration and graphical placement of each compound included in a PFAS containing compound according to the framework. Such PFAS patterns may be provided as output to user devices and may ease interpretation of PFAS datasets by providing a consistent visual pattern for interpretation. These PFAS patterns may be thought of as a chemical fingerprint of the PFAS compound, and may include data on the hundreds of PFAS precursors used in PFAS modeling.

It is also difficult to predict how PFAS compounds or PFAS containing products transport in and interact with an environment. For example, simulating how PFAS compounds or PFAS containing products are affected by the processes of advection, dispersion, adsorption, transformation, and other factors is difficult due to the complex nature of PFAS compounds. Existing models of PFAS compounds do not generally account for transformation of the thousands of possible PFAS precursors and instead focus on a small subset of approximately 10 to 20 PFAS compounds. Further, current models do not allow modeling of total organofluorine (TOF) as an indicator of the total PFAS analytical mass. The PFAS model disclosed herein may utilize computer aided modeling supporting input of PFAS data and other environmental factors to provide output of estimated PFAS compound concentrations in three dimensions within an environment under various time scenarios. The PFAS model disclosed herein allows modeling of the transformation of hundreds of PFAS compounds and TOF in three-dimensional space and over time.

Determining which PFAS containing compounds or products caused a particular PFAS concentration pattern in an environment is also a difficult task due to the complexities of PFAS compounds and their behavior over time in the environment. Current PFAS modeling does not utilize machine learning models for PFAS product source identification. As current PFAS modeling generally focuses on a small subset of PFAS compounds, PFAS product source identification modeling based on current PFAS modeling would likely be inaccurate, as such modeling would not account for how a larger number (e.g., hundreds to thousands) of PFAS compounds transform and attenuate once released into the environment. The PFAS model described herein may utilize a PFAS product model including a machine learning model trained or generated using simulated PFAS and environment data. The machine learning model predicts which PFAS containing products or compounds could have resulted in the respective PFAS concentrations identified in the environment. The machine learning model may be trained using datasets generated by other features of the PFAS model and, accordingly, may take into account larger number of PFAS containing products or compounds, improving accuracy of the model.

The various components and functionalities of the PFAS model described above may be used in conjunction with one another or independently to analyze PFAS compounds and PFAS containing compounds. For example, a simulation component of the PFAS model may generate training datasets for training the machine learning model. In some examples, the simulation component and/or the machine learning model may provide, as part of their respective outputs, PFAS patterns for PFAS compounds of interest (e.g., those observed in an environment, predicted to be present in an environment, and/or suspected to have caused observed PFAS concentrations in an environment).

The PFAS model disclosed herein may utilize environmental data relating to, for example, an environment where PFAS compounds or PFAS containing products are located. Environmental data may include, for example, observed concentrations of PFAS compounds in an environment, data about various conditions in the environment (e.g., moisture, temperature fluctuations, and the like), and/or data about the type of environment or different types of environments within an environment (e.g., soil composition, water, air, and the like). The PFAS model may further be able to account for various simulation variables which may include environmental data, a time period of the simulation, expected conditions in the environment over time, and the like). Accordingly, predictions and simulations created using the various components of the PFAS model may be specific to an environment of interest.

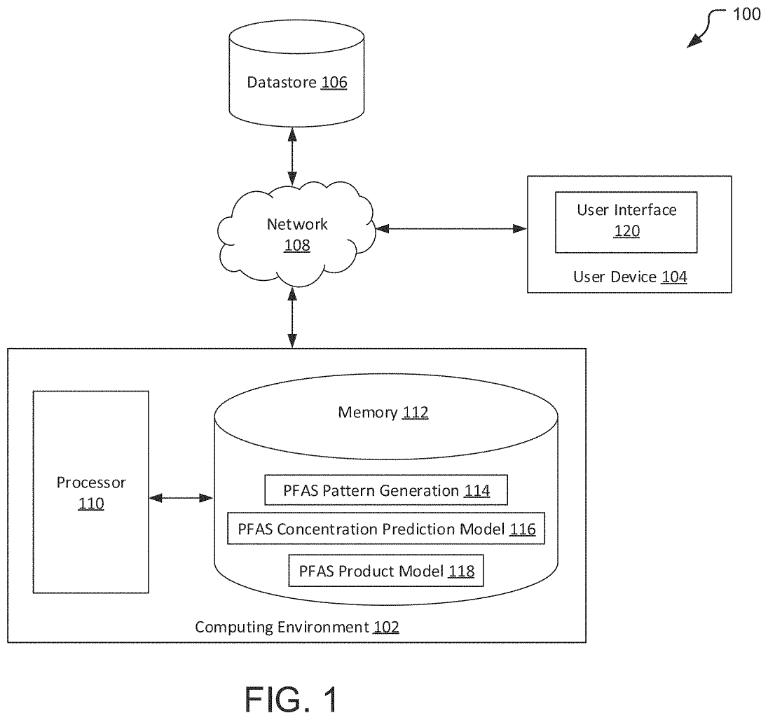

Various embodiments of the present disclosure will be explained below in detail with reference to the accompanying drawings. Other embodiments may be utilized, and structural, logical and electrical changes may be made without departing from the scope of the present disclosure. FIG. 1 illustrates an example system 100 providing PFAS pattern generation, a PFAS concentration prediction model, and a PFAS product model in accordance with some embodiments of the disclosure. A PFAS model 102 may include memory 122 storing instructions for PFAS pattern generation 114, a PFAS concentration prediction model 116, and a PFAS product model 118. Such instructions, when executed by one or more processors 110 of the PFAS model 102 implement various functionalities of the PFAS model 102. The computing environment 102 may communicate with one or more datastores 106 via a network 108. One or more user devices 104 may communicate with the PFAS model 102 via the network 108 to utilize PFAS pattern generation 114, the PFAS concentration prediction model 116, and/or the PFAS product model 118. In some examples, a user interface 120 at the user device 104 may be used to provide information (e.g., input data) to, and display information (e.g., outputs) from the PFAS model 102.

Generally, the user device 104 may be a device belonging to an end user accessing the system 100. In various embodiments, multiple user devices 104 may be provided with access to the PFAS model 102 to utilize PFAS pattern generation 114, the PFAS concentration prediction model 116, and/or the PFAS product model 118. Where multiple user devices 104 access the PFAS model 102, the user devices 104 may be provided with varying permission, settings, and the like, and may be authenticated by an authentication service prior to access the PFAS model 102. In various implementations, the user device 104 and/or additional user devices may be implemented using any number of computing devices included, but not limited to, a desktop computer, a laptop, tablet, mobile phone, smart phone, wearable device (e.g., AR/VR headset, smart watch, smart glasses, or the like), smart speaker, vehicle (e.g., automobile), or appliance. Generally, the user device 104 may include one or more processors, such as a central processing unit (CPU) and/or graphics processing unit (GPU). The user device 104 may generally perform operations by executing executable instructions (e.g., software) using the processors.

In some examples, a user interface 120 at the user device 104 may be used to provide information (e.g., input data) to, and display information (e.g., outputs) from the PFAS model 102. In various embodiments, the user interface 120 may be implemented as a React, Javascript-based interface for interaction with the PFAS model 102. The user interface 120 may also access various components of the PFAS model 102 locally at the user device 104, through webpages, one or more applications at the user device 104, or using other methods. The user interface 120 may also be used to display output from the PFAS model 102, such as PFAS patterns, data about PFAS transformation and/or degradation, PFAS compound or PFAS containing product identification, and other data and/or visual output generated by the PFAS model 102.

The network 108 may be implemented using one or more of various systems and protocols for communications between computing devices. In various embodiments, the network 108 or various portions of the network 108 may be implemented using the Internet, a local area network (LAN), a wide area network (WAN), and/or other networks. In addition to traditional data networking protocols, in some embodiments, data may be communicated according to protocols and/or standards including near field communication (NFC), Bluetooth, cellular connections, and the like. Various components of the system 100 may communicate using different network protocols or communications protocols based on location. For example, components of the PFAS model 102 may be hosted within a cloud computing environment and may communicate with each other using communication and/or network protocols used by the cloud computing environment.

The system 100 may include one or more datastores 106 storing various information and/or data including, for example, PFAS data, frameworks for representing PFAS data, and the like. For example, the datastore may include data about concentrations of PFAS compounds within various PFAS containing products and/or other information about PFAS compounds obtained from studies, published literature, and/or previous models created by the PFAS model 102. In some examples, the datastore 106 may further store training data sets used to train or generate the machine learning model, reference data for various environmental scenarios, stored user data, and the like.

In various implementations, the PFAS model 102 may include or utilize one or more hosts or combinations of compute resources, which may be located, for example, at one or more servers, cloud computing platforms, computing clusters, and the like. Generally, the PFAS model 102 is implemented by compute resources including hardware for memory 112 and one or more processors 110. For example, the PFAS model 102 may utilize or include one or more processors, such as a CPU, GPU, and/or programmable or configurable logic. In some embodiments, various components of the PFAS model 102 may be distributed across various computing resources, such that the components of the PFAS model 102 communicate with one another through the network 108 or using other communications protocols. For example, in some embodiments, the PFAS model 102 may be implemented as a serverless service, where computing resources for various components of the PFAS model 102 may be located across various computing environments (e.g., cloud platforms) and may be reallocated dynamically and automatically according to resource usage of the PFAS model 102. In various implementations, the PFAS model 102 may be implemented using organizational processing constructs such as functions implemented by worker elements allocated with compute resources, containers, virtual machines, and the like.

The memory 112 may include instructions for various functions of the PFAS model 102, which, when executed by processor 110, preform various functions of the PFAS model 102. Similar to the processor 110, memory resources utilized by the PFAS model 102 may be distributed across various physical computing devices.

The PFAS model 102 may receive data, instructions, and other communications from the user device 104 (and other user devices) to perform various modeling functions related to PFAS containing compounds. For example, the user device 104 may provide environmental data and/or PFAS compound data to the PFAS model 102 and may receive, from the PFAS model 102, PFAS patterns (e.g., 3D fingerprints) for PFAS compounds or PFAS containing products in the environment. The PFAS model 102 may utilize instructions stored at memory 112 for PFAS pattern generation 114 to generate PFAS patterns based on the provided environmental data and/or PFAS compound data. Such patterns may provide a visual graph or chart depicting the concentration of various PFAS compounds within a PFAS containing product. For example, the PFAS model 102 may receive a selection of a type of PFAS containing product from the user device 104 and may retrieve data about the chemical makeup of the PFAS containing product from datastore 106. In some examples, the PFAS pattern may be based on a PFAS framework utilized by the PFAS model 102 and the PFAS model may further retrieve the framework from the datastore 106. PFAS pattern generation 114 may then create the PFAS pattern using the retrieved data. PFAS pattern generation 114 may further transmit the PFAS pattern to the user device 104 for display at the user interface 120.

In some examples, the user device 104 may provide environmental data and/or PFAS compound data to the PFAS model 102 and may receive, from the PFAS model 102, data about PFAS compounds or PFAS containing products in the environment after transformation and/or degradation of original PFAS compounds or products. The PFAS model 102 may utilize instructions stored at memory 112 for the PFAS concentration prediction model 116 to generate data about PFAS containing compounds in the environment after degradation of the original PFAS compounds. The PFAS concentration prediction model 116 may calculate and predict how individual PFAS compounds and total organofluorine (TOF) will be affected by the processes of advection, dispersion, adsorption, transformation, and attenuation over time and in three-dimensional space. The PFAS concentration model 116 may allow input of thousands of individual PFAS compounds, environmental factors that affect PFAS mobility, transformation, and attenuation (e.g., attenuation zones at different distances from the source, geochemical conditions, seepage velocity, retardation, dispersivity, source zone width and thickness, and the like), time, the spatial domain of interest, and other relevant conditions that may affect predicted PFAS concentration changes in the environment (e.g., microbial population interactions or other retardation, transformation, or attenuation factors).

Based on the provided parameters, the PFAS concentration model 116 may predict resulting PFAS concentrations over three-dimensional space and time according to high, median, and low values corresponding to the respective ranges of transformation and attenuation rates provided to the PFAS concentration model 116. The PFAS concentration model 116 may be developed within a spreadsheet framework or may use other methods (e.g., machine learning) to predict PFAS concentrations. The PFAS concentration model 116 may allow for incorporation of additional PFAS compounds as new data or literature references become available. The PFAS concentration model 116 may predict PFAS concentrations in a variety of environments and distinct areas of environments including, for example, in soil, groundwater, surface water, and air.

In some examples, the PFAS concentration model 116 may provide, as output, further information about predicted PFAS concentrations within a three-dimensional area. Such output may be provided graphically, for display, for example, at the user interface 120 of the user device 104. In some examples, such output may be compared with observed PFAS concentrations within an environment to help determine which PFAS containing products resulted in the observed concentrations.

The user device 104 may also provide environmental data to the PFAS model 102 and may receive, from the PFAS model 102, identification of PFAS containing compounds in the environment. The PFAS model 102 may utilize instructions stored at memory 112 for the PFAS product model 118 to generate identification of PFAS containing compounds in the environment based on the provided environmental data. The PFAS product model 118 may be a machine learning model trained or generated to identify PFAS chemical patterns associated with the environmental release of different PFAS containing products. The machine learning model of the PFAS product model 118 may be any type of machine learning model or combinations of machine learning models using predictive techniques, such as neural networks, support vector machines, decision trees, classifiers, and the like. In some examples, the PFAS product model 118 may be updated over time by updating a training dataset using output from the PFAS product model 118 and/or through other feedback methods providing additional input and/or training to the machine learning model. The PFAS product model 118 allows for prediction or identification of PFAS containing products that observed PFAS concentrations result from. This allows confirmation as to whether observed PFAS contamination is from a known release at the site of interest, or if observed PFAS contamination has resulted from a different PFAS containing product that has migrated onto the site of interest.

Though not depicted in FIG. 1, the PFAS model 102 may include or interact with additional software or hardware components for various functions. For example, an authorization service may be utilized to connect user devices to the PFAS model 102. Users may sign into the PFAS model 102 and may be directed to an authorization service. Upon authorization, the user may access data and use the user interface 120 to interact with the PFAS model 102. In another example, the PFAS model 102 may be accessible by means other than the user interface 120. For example, various users may connect to the PFAS model 102 through, for example, command line instructions, and may provide commands to the PFAS model 102 through scripts, text commands, or means other than a user interface 120. Additional services and/or applications may be utilized by the PFAS model 102 to perform functions such as querying databases, communicating between components, managing compute resources delegated to the PFAS model 102, and other functions.

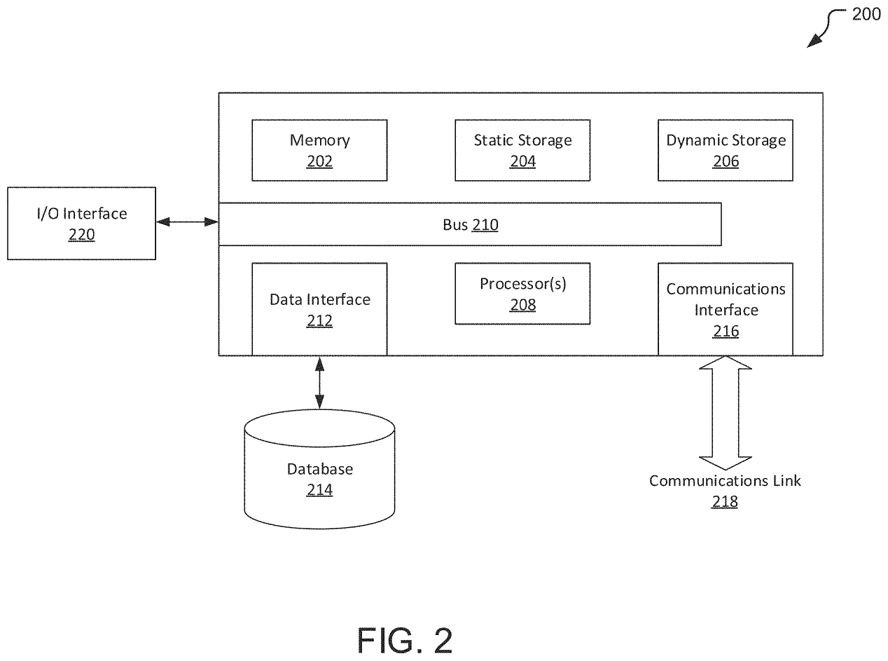

FIG. 2 is a schematic diagram of a computing system 200 which may be used to implement various embodiments in the examples described herein. For example, processor 110 and memory 112 may be located at one or several computing systems 200. In various embodiments, user device 104 is also implemented by a computing system 200. This disclosure contemplates any suitable number of computing systems 200. For example, a computing system 200 may be a server, a desktop computing system, a mainframe, a mesh of computing systems, a laptop or notebook computing system, a tablet computing system, or a combination of two or more of these. Where appropriate, the computing system 200 may include one or more computing systems; be unitary or distributed; span multiple locations; span multiple machines; span multiple data centers; or reside in a cloud, which may include one or more cloud components in one or more networks.

Computing system 200 includes a bus 210 (e.g., an address bus and a data bus) or other communication mechanism for communicating information, which interconnects subsystems and devices, such as processor 208, memory 202 (e.g., RAM), static storage 204 (e.g., ROM), dynamic storage 206 (e.g., magnetic or optical), communications interface 216 (e.g., modem, Ethernet card, a network interface controller (NIC) or network adapter for communicating with an Ethernet or other wire-based network, a wireless NIC (WNIC) or wireless adapter for communicating with a wireless network, such as a WI-FI network), input/output (I/O) interface 220 (e.g., a keyboard, keypad, mouse, microphone). In particular embodiments, the computing system 200 may include one or more of any such components.

In particular embodiments, processor 208 includes hardware for executing instructions, such as those making up a computer program. The processor 208 circuitry includes circuitry for performing various processing functions, such as executing specific software for perform specific calculations or tasks. In particular embodiments, I/O interface 220 includes hardware, software, or both, providing one or more interfaces for communication between computing system 200 and one or more I/O devices. Computing system 200 may include one or more of these I/O devices, where appropriate. One or more of these I/O devices may enable communication between a person and computing system 200.

In particular embodiments, communications interface 216 includes hardware, software, or both providing one or more interfaces for communication (such as, for example, packet-based communication) between computing system 200 and one or more other computer systems or one or more networks. One or more memory buses (which may each include an address bus and a data bus) may couple processor 208 to memory 202. Bus 210 may include one or more memory buses, as described below. In particular embodiments, one or more memory management units (MMUs) reside between processor 208 and memory 202 and facilitate accesses to memory 202 requested by processor 208. In particular embodiments, bus 210 includes hardware, software, or both coupling components of computing system 200 to each other.

According to particular embodiments, computing system 200 performs specific operations by processor 208 executing one or more sequences of one or more instructions contained in memory 202. For example, instructions for PFAS pattern generation 114, the PFAS concentration prediction model 116, and the PFAS product model 118 may be contained in memory 202 and may be executed by the processor 208. Such instructions may be read into memory 202 from another computer readable/usable medium, such as static storage 204 or dynamic storage 206. In alternative embodiments, hard-wired circuitry may be used in place of or in combination with software instructions. Thus, particular embodiments are not limited to any specific combination of hardware circuitry and/or software. In one embodiment, the term “logic” shall mean any combination of software or hardware that is used to implement all or part of particular embodiments disclosed herein.

The term “computer readable medium” or “computer usable medium” as used herein refers to any medium that participates in providing instructions to processor 208 for execution. Such a medium may take many forms, including but not limited to, nonvolatile media and volatile media. Non-volatile media includes, for example, optical or magnetic disks, such as static storage 204 or dynamic storage 206. Volatile media includes dynamic memory, such as memory 202.

Computing system 200 may transmit and receive messages, data, and instructions, including program, e.g., application code, through communications link 218 and communications interface 216. Received program code may be executed by processor 208 as it is received, and/or stored in static storage 204 or dynamic storage 206, or other storage for later execution. A database 214 may be used to store data accessible by the computing system 200 by way of data interface 212.

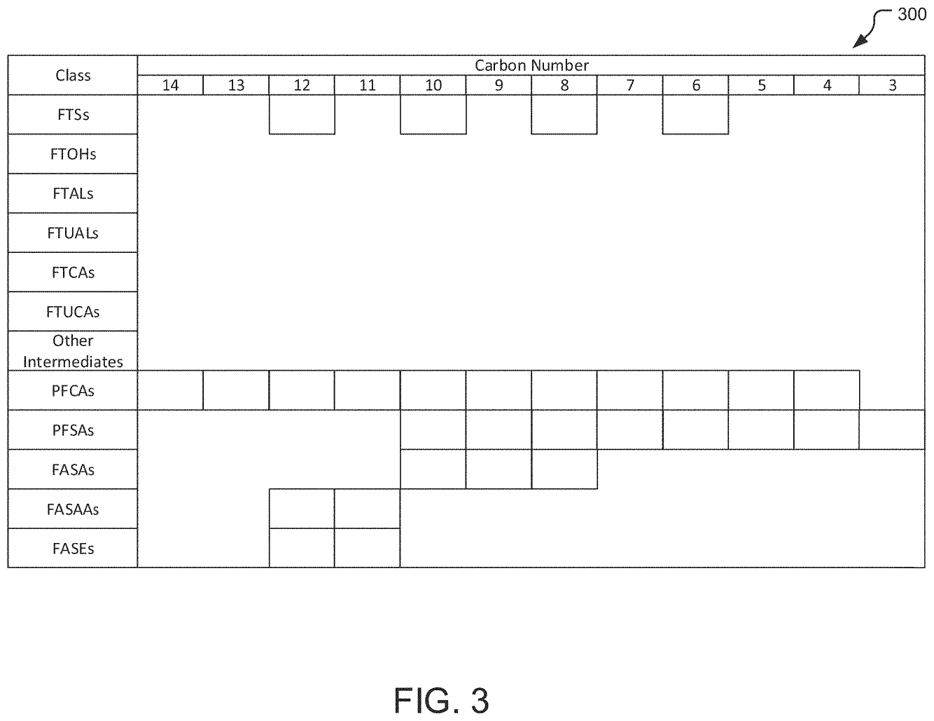

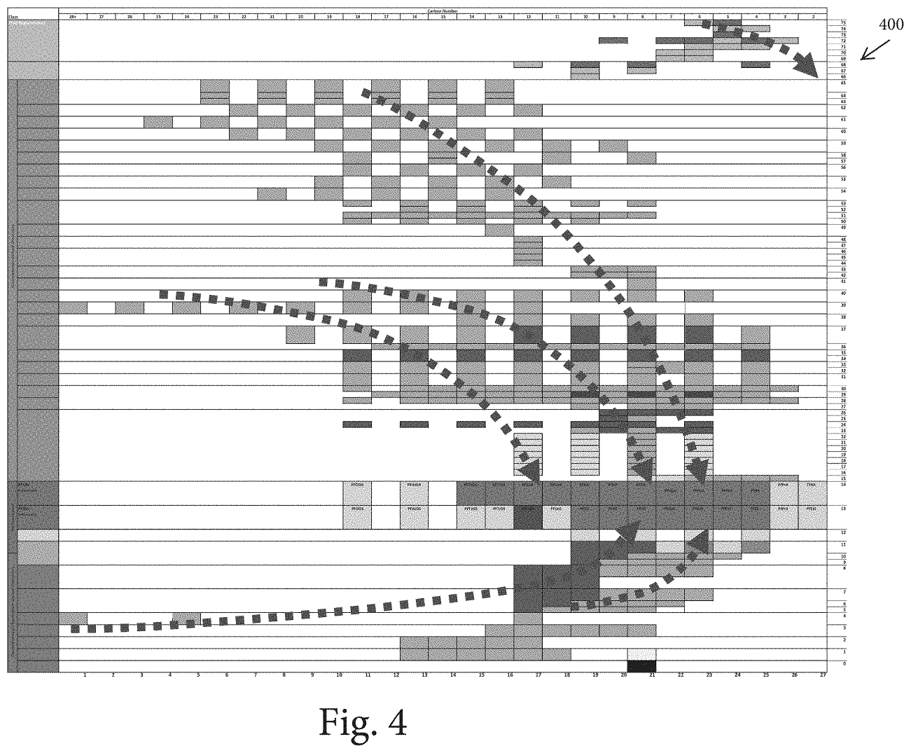

FIG. 3 shows an example framework 300 for representing PFAS containing compounds used in accordance with various embodiments of the disclosure. FIG. 4 shows an additional example framework 400 for representing PFAS containing compounds used in accordance with various embodiments of the disclosure. The framework 300 may be a subset of the framework 400 (e.g., a simplified version or showing a portion of the framework 400). As shown, the framework 300 organizes compounds based on carbon number and class of PFAS compound. The framework 400 similarly organizes compounds based on carbon number (number of carbon atoms in a compound) and by the respective PFAS chemical subclass, group, and subgroup (e.g., polymer versus non-polymer, perfluorinated versus polyfluorinated, perfluoroalkyl carboxylic acids, perfluoroalkane sulfonic acids, perfluoroalkane sulfonamides, fluorotelomer sulfonic acids, and the like). The framework 400 further organizes PFAS compounds by manufacturing process and/or product type. The PFAS compounds may be ordered according to how to the compounds transform from one to another in the environment, with arrows between various compounds being color-coded according to the environmental condition under which the transformation reactions occur (e.g., aerobic versus anaerobic pathways). The framework 400 as a whole may be color coded to indicate other characteristics or groupings of PFAS compounds, such as chemical class, perfluorinated versus polyfluorinated compounds, volatility, compounds reported under standard analytical methods versus non-standard analytical methods, and other such identifying information.

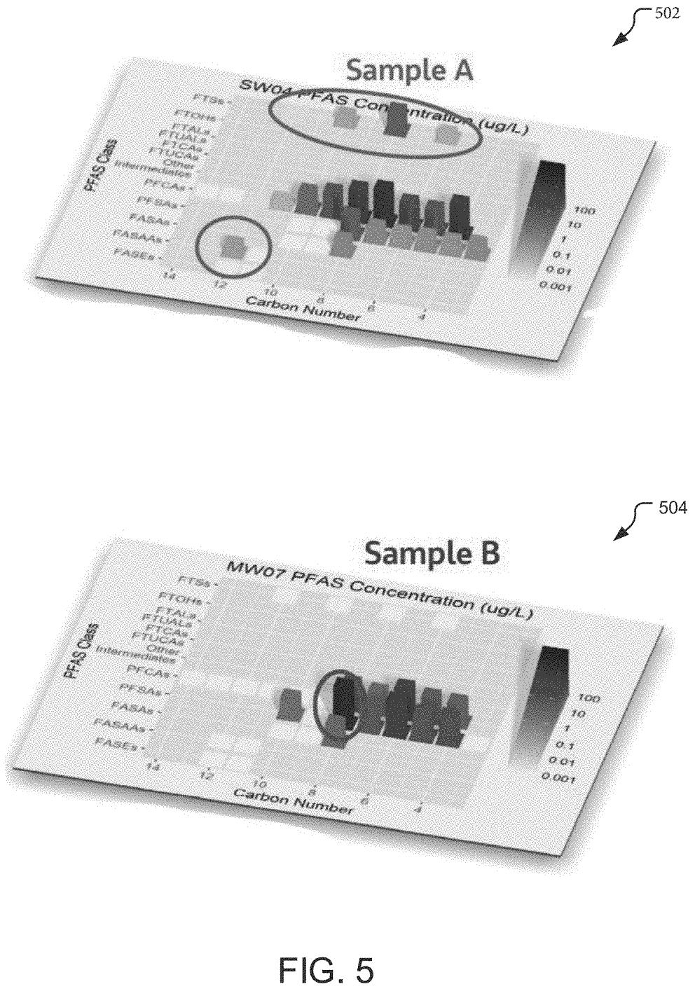

FIG. 5 shows example PFAS patterns 502 and 504 generated in accordance with various embodiments of the disclosure. The PFAS patterns 502 and 504 may be based on the framework utilized by the PFAS model 102. For example, the PFAS patterns 502 and 504 include plots of concentrations of individual PFAS compounds in a PFAS containing product or compound according to the order of the compounds in the framework 300. The PFAS concentrations may be plotted by color coding, bar charts, or other similar graphical means to produce a visual pattern of concentrations in an order consistent with the framework 300 or 400. In some examples, PFAS patterns may be used to distinguish between similar PFAS compounds to help to identify a source of the compound. For example, PFAS patterns 502 and 502 may represent samples with identical total molar concentrations, which may be difficult to distinguish. However, as shown, the two samples include different concentrations of different PFAS compounds and, accordingly, can be distinguished visually using the PFAS patterns 502 and 504.

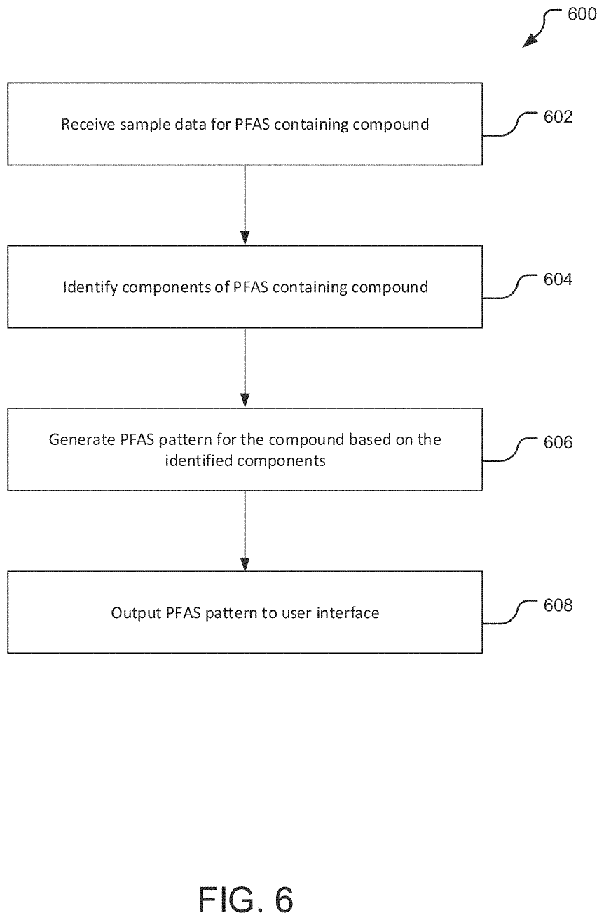

FIG. 6 is a flow diagram of an example process 600 for generating a PFAS pattern in accordance with various embodiments of the disclosure. At operation 602, the PFAS model 102 receives sample data for a PFAS containing compound. The sample data may include, for example, measurements of PFAS compound concentrations at various locations within an environment. In some examples, sample data may include the identity of a known PFAS containing product. In various examples, the sample data may be received from the user device 104. For example, the sample data may be provided through a user interface 120 at the user device or otherwise transmitted to the PFAS model 102. For example, the data may be transmitted from the user device 104 to the PFAS model 102 in tab delimited or comma delimited (e.g., csv) format. In some examples, data may be transmitted directly from a measurement device to the PFAS model 102.

At operation 604, the PFAS model 102 identifies components of the PFAS containing compound. In some examples, the PFAS model 102 may determine whether the received sample data includes sufficient information to generate a PFAS pattern. Where the sample data does not include sufficient information (e.g., concentrations of PFAS compounds within a PFAS containing product or in the environment), the PFAS model 102 may return a message to the user device 104 asking for additional data and/or may access the datastore 106 to retrieve additional data. For example, where the PFAS model 102 is used to generate a PFAS pattern or fingerprint for a known PFAS containing product, the datastore 106 may include data on the relative or absolute concentrations of various PFAS compounds within the product.

At operation 606, the PFAS model 102 generates a PFAS pattern for the compound based on the identified components. PFAS pattern generation 114 may use the framework as a starting point to generate the PFAS pattern and may then plot the various PFAS compound concentrations within the framework. In some examples, the concentrations may be plotted to create a three dimensional pattern, as shown in FIG. 5. In some examples, the concentrations may be otherwise differentiated within the framework based on the framework to create a three dimensional pattern. For example, the concentrations may be color coded, textured, given numeric values, or otherwise differentiated based on concentration.

At operation 608, the PFAS model 102 outputs the PFAS pattern to a user interface. The PFAS model 102 may output the PFAS pattern to the user interface 120 of the user device 104. In some examples, the PFAS model 102 may, instead or additionally, transmit the PFAS pattern to one or more other computing devices for display and/or analysis. In some examples, the PFAS model 102 may store the PFAS pattern at datastore 106 for later use.

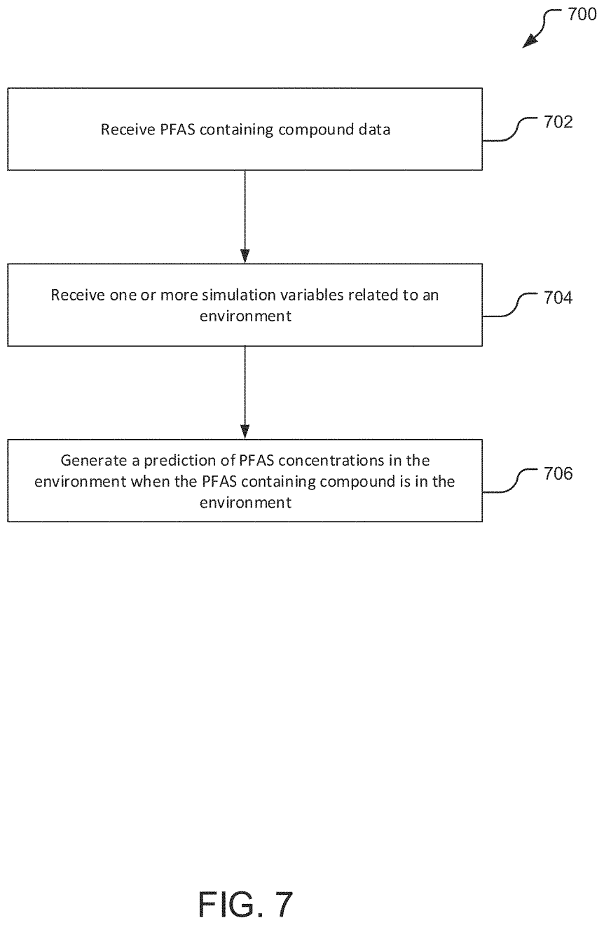

FIG. 7 is a flow diagram of an example process 700 for generating predictions of future PFAS concentrations in accordance with various embodiments of the disclosure. At operation 702, the PFAS model 102 receives PFAS containing product or compound data. The PFAS containing product or compound data may include a PFAS pattern for a PFAS containing product, expected concentrations of PFAS compounds within a PFAS containing product, and the like. In some examples, the PFAS containing product data may be the name or other identifier of a PFAS containing product. The PFAS model 102 may then locate or access other data, such as concentrations of PFAS compounds in the PFAS containing product.

At operation 704, the PFAS model 102 receives one or more simulation variables related to an environment. Simulation variables may include, in various examples, a time duration for the simulation, whether transformation is aerobic or anaerobic, temperature, pH, distance from a source of the PFAS containing product, seepage velocity, and other variables related to the environment. In some examples, the PFAS model 102 may use default values for the simulation variables when no additional values are provided. The PFAS model 102 may receive the simulation variables from the user device 104 (e.g., a user may enter the simulation variables via the user interface 120) or from another source or device. For example, some simulation variables may be directly measured from the environment. In some examples, the PFAS model 102 may look up some simulation variables based on the location or other provided characteristics of the environment. For example, a user may provide a location of the environment and the PFAS model 102 may use the location to determine a value for soil pH (e.g., by querying the datastore 106 or from an external data source).

At operation 706, the PFAS model 102 generates a prediction of PFAS concentrations in the environment when the PFAS containing compound is in the environment. The prediction of PFAS concentrations may be generated by the PFAS concentration prediction model 116 based on the given environmental variables and over the given timeframe. The prediction may include expected concentration of various PFAS compounds at different three-dimensional locations within the given environment. In some examples, such data may be provided graphically, such as through a three-dimensional representation of the environment transmitted from the PFAS concentration prediction model 116 to the user device 104 for display at the user interface 120 of the user device. In some examples, the PFAS concentration prediction model 116 may communicate its results to PFAS pattern generation 114, which may generate expected patterns for various locations in the environment based on the predictions of the PFAS concentration prediction model 116. Such patterns may be transmitted to the user device 104 and may be displayed via the user interface 120.

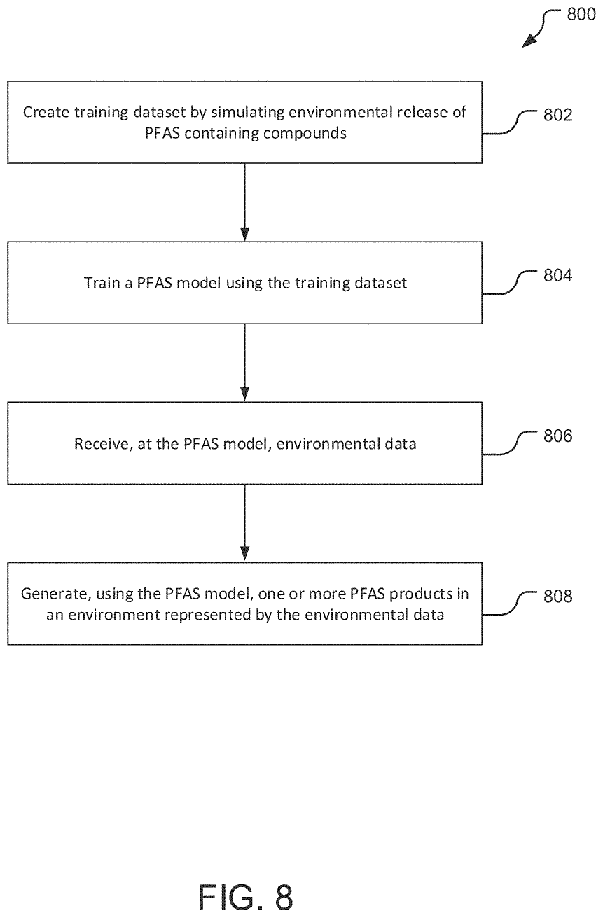

FIG. 8 is a flow diagram of an example process 800 for generating and using a PFAS product model in accordance with various embodiments of the disclosure. At operation 802, the PFAS model 102 creates a training dataset by simulating environmental release of PFAS containing compounds. In various examples, the PFAS model 102 may simulate environmental release and attenuation of PFAS compounds using the process 700 described with respect to FIG. 7. The training dataset may be designed to cover various environmental conditions and types of PFAS containing products to create a broader model. In some examples, the training dataset may include additional data points gathered by direct measurement. For example, where the source (e.g., a PFAS containing product) of PFAS concentration within an environment is known, measured PFAS concentrations throughout the environment may be added to the training dataset.

At operation 804, the PFAS model 102 trains a PFAS product model 118 using the training dataset. The training may differ based on the type of machine learning model used, but generally uses the training data set to generate labeled observations to train the model. In some examples, a portion of the training data set may be used to verify the training of the model by providing input to the model after training and comparing the output of the model to known values.

At operation 806, the PFAS product model 118 receives environmental data. The environmental data may include measured PFAS compound concentrations within an environment, measurements (e.g., size measurements) of the environment or various components of the environment, characteristics of components of the environment (e.g., temperature, soil pH, elevation, and the like), and other data regarding the environment. The environmental data may be transmitted by the user device 104, such as by being provided as input via the user interface 120. In some examples, environmental data may be provided by multiple computing devices. For example, PFAS compound concentrations may be provided directly to the PFAS product model 118 from a measuring device. Measurements of the environment and environmental characteristics may then be separately provided via the user device 104. In some examples, some environmental data may further be determined by the PFAS model 102 based on other data provided to the PFAS model 102. For example, the user device 104 may provide a geographic location of the environment, and the PFAS model 102 may then use the provided location to determine environmental conditions by, for example, querying an external data source providing atmospheric or soil conditions for a geographic region.

At operation 808, the PFAS model 102 generates, using the PFAS product model 118, identification of one or more PFAS products in an environment represented by the environmental data. The identification of the one or more PFAS products may, in some examples, include additional elements, such as an estimated location of release of the PFAS containing products, amount of PFAS containing products released, and duration of release of the PFAS containing products. For example, the PFAS product model 118 may determine that, based on the PFAS compound concentration and environmental data, a certain PFAS containing product was likely released 1000 ft from the environment of interest over a period of two years. In some examples, the PFAS product model 118 may provide alternative scenarios and may provide a likelihood of correctness of one or more predictions. The PFAS model 102 may further transmit the predictions to one or more user devices 104, store the predictions at one or more datastores 106, or perform other operations with the predictions as requested by the user.

Using the above methods, accurate modeling of PFAS compound concentrations in an environment are possible by taking into account hundreds or thousands of PFAS compounds and how the compounds are likely to attenuate and evolve over time. For example, the PFAS model described above may track and utilize information about how PFAS compounds transform along a flow path over time. Accordingly, utilizing the PFAS model disclosed herein, PFAS compounds and their effects on the environment can be accurately modeled over time, assisting in remediation efforts.

The technology described herein may be implemented as logical operations and/or modules in one or more systems. The logical operations may be implemented as a sequence of processor-implemented steps directed by software programs executing in one or more computer systems and as interconnected machine or circuit modules within one or more computer systems, or as a combination of both. Likewise, the descriptions of various component modules may be provided in terms of operations executed or effected by the modules. The resulting implementation is a matter of choice, dependent on the performance requirements of the underlying system implementing the described technology. Accordingly, the logical operations making up the embodiments of the technology described herein are referred to variously as operations, steps, objects, or modules. Furthermore, it should be understood that logical operations may be performed in any order, unless explicitly claimed otherwise or a specific order is inherently necessitated by the claim language.

In some implementations, articles of manufacture are provided as computer program products that cause the instantiation of operations on a computer system to implement the procedural operations. One implementation of a computer program product provides a non-transitory computer program storage medium readable by a computer system and encoding a computer program. It should further be understood that the described technology may be employed in special purpose devices independent of a personal computer.

The above specification, examples and data provide a complete description of the structure and use of exemplary embodiments of the invention as defined in the claims. Although various embodiments of the claimed invention have been described above with a certain degree of particularity, or with reference to one or more individual embodiments, it is appreciated that numerous alterations to the disclosed embodiments without departing from the spirit or scope of the claimed invention may be possible. Other embodiments are therefore contemplated. It is intended that all matter contained in the above description and shown in the accompanying drawings shall be interpreted as illustrative only of particular embodiments and not limiting. Changes in detail or structure may be made without departing from the basic elements of the invention as defined in the following claims.

1. A method comprising:
creating a training dataset of per and polyfluoroalkyl substances (PFAS) compound environmental release over time using a computer aided simulation of environmental release of a set of PFAS containing products;
training a machine learning model using the training dataset;
receiving, at the trained machine learning model, environmental data relating to an environment and PFAS concentration data representing PFAS compound concentrations within the environment; and
generating, using the trained machine learning model, identification of one or more PFAS containing products likely to have caused the PFAS compound concentrations within the environment.

2. The method of claim 1, wherein the environmental data and the PFAS concentration data are received from a user device, the method further comprising transmitting the identification of the one or more PFAS containing products to the user device.

3. The method of claim 1, further comprising:
generating a graphical representation of the environment based on the identification of the one or more PFAS containing products.

4. The method of claim 1, wherein the environmental data includes one or more environmental conditions of the environment.

5. The method of claim 1, wherein the computer aided simulation accounts for one or more of environmental temperature, environmental pH, distance from a source of the PFAS containing product, and seepage velocity.

6. The method of claim 1, further comprising:
generating a likelihood of correctness of the identification of the one or more PFAS containing products likely to have caused the PFAS compound concentrations within the environment.

7. The method of claim 1, wherein the PFAS concentration data represents PFAS concentrations in three dimensions with relation to a source.

8. A system comprising:
one or more processors; and
memory encoded with instructions which, when executed by the one or more processors, cause the system to:
create a training dataset of per and polyfluoroalkyl substances (PFAS) compound environmental release over time using a computer aided simulation of environmental release of a set of PFAS containing products;
receive, at the trained machine learning model, environmental data relating to an environment and PFAS concentration data representing PFAS compound concentrations within the environment; and
generate, using the trained machine learning model, identification of one or more PFAS containing products likely to have caused the PFAS compound concentrations within the environment.

9. The system of claim 8, wherein the instructions further cause the system to:
receive the environmental data and the PFAS concentration data from a user device; and
transmit the identification of the one or more PFAS containing products to the user device.

10. The system of claim 8, wherein the instructions further cause the system to:
generate a graphical representation of the environment based on the identification of the one or more PFAS containing products.

11. The system of claim 8, wherein the environmental data includes one or more environmental conditions of the environment.

12. The system of claim 8, wherein the computer aided simulation accounts for one or more of environmental temperature, environmental pH, distance from a source of the PFAS containing product, and seepage velocity.

13. The system of claim 8, wherein the instructions further cause the system to:
generate a likelihood of correctness of the identification of the one or more PFAS containing products likely to have cause the PFAS compound concentrations within the environment.

14. The system of claim 8, wherein the PFAS concentration data represents PFAS concentrations in three dimensions with relation to a source.

15. One or more non-transitory computer readable media encoded with instructions which, when executed by one or more processors, cause the one or more processors to:
create a training dataset of per and polyfluoroalkyl substances (PFAS) compound environmental release over time using a computer aided simulation of environmental release of a set of PFAS containing products;
train a machine learning model using the training dataset;
receive, at the trained machine learning model, environmental data relating to an environment and PFAS concentration data representing PFAS compound concentrations within the environment; and
generate, using the trained machine learning model, identification of one or more PFAS containing products likely to have cause the PFAS compound concentrations within the environment.

16. The one or more non-transitory computer readable media of claim 15, wherein the instructions further cause the one or more processors to:
receive the environmental data and the PFAS concentration data from a user device; and
transmit the identification of the one or more PFAS containing products to the user device.

17. The one or more non-transitory computer readable media of claim 15, wherein the instructions further cause the one or more processors to:
generate a graphical representation of the environment based on the identification of the one or more PFAS containing products.

18. The one or more non-transitory computer readable media of claim 15, wherein the computer aided simulation accounts for one or more of environmental temperature, environmental pH, distance from a source of the PFAS containing product, and seepage velocity.

19. The one or more non-transitory computer readable media of claim 15, wherein the instructions further cause the one or more processors to:
generate a likelihood of correctness of the identification of the one or more PFAS containing products likely to have caused the PFAS compound concentrations within the environment.

20. The one or more non-transitory computer readable media of claim 15, wherein the PFAS concentration data represents PFAS concentrations in three dimensions with relation to a source.
